(19) United States
(12) Patent Application Publication
(10) Pub. No.: US 2017/0008785 A1
(43) Pub. Date: Jan. 12, 2017

(54) System and Method for Treating Water with a Biological Based Biocide

(71) Applicant: NCH Corporation, Irving, TX (US)

(72) Inventors: Adrian J. Denvir, Richardson, TX (US); David F. Vela, Irving, TX (US); Matthew C. Holloway, Fort Worth, TX (US); Angela Hutson, Denton, TX (US)

(21) Appl. No.: 15/204,119

(22) Filed: Jul. 7, 2016

Related U.S. Application Data
(60) Provisional application No. 62/189,447, filed on Jul. 7, 2015.

(51) Int. Cl. C02F 3/34; C12M 1/34; C02F 3/02

(52) U.S. Cl. CPC C02F 3/34; C02F 3/02; C12M 41/26; C12M 41/12; C02F 2209/06; C02F 2209/22; C02F 2209/02; C02F 2203/002; C02F 2209/24; C02F 2303/20; C02F 2303/04; C02F 2103/023

(57) ABSTRACT

A system and method of treating water systems with amoebas produced on-site to reduce biological contaminants, such as algae, bacteria and biofilms, without requiring the use of, or reducing the amount of, chemical treatments that produce harmful by-products. The system and method comprise generating an amoeba treatment solution using an on-site biogenerator and discharging the treatment solution in the water system at predetermined discharge intervals. The operating parameters of the biogenerator are monitored and controlled to maintain one or more operating conditions, such as dissolved gas levels, temperature, or pH inside a growth tank within desired ranges. An amount of amoeba starter material sufficient to supply the biogenerator for a prolonged treatment cycle, such as 30 days, is provided and stored in a temperature controlled environment near the biogenerator to maintain the viability of the amoebas prior to generating each dose of treatment solution.

### CROSS REFERENCE TO RELATED APPLICATION

This application claims the benefit of U.S. Provisional Application Ser. No. 62/189,447 filed on Jul. 7, 2015.

### BACKGROUND OF THE INVENTION

1. Field of the Invention

This invention relates to a system and method for treating water, such as industrial process water, wastewater, and cooling tower water, with amoebas as a biological based biocide to remove algae, biofilms and harmful bacteria.

2. Description of Related Art

In the United States today there is a growing concern about the scarcity of water and the continuing drought problems and how this will impact food prices and energy production. The World Economic Forum has identified some of the critical facts that relate to the increasing demand for water, energy, and food over the next 20 years. Over the next two decades the population is expected to increase to 8 billion and this will be accompanied with economic growth of around 6% in developing nations and 3% in developed countries. As a result there will be an expected increase in urbanization of 50%. To meet these growing demands the agricultural sector will need to increase production by 70%-100% and the global energy consumption will rise. This equates to a demand that is almost double our current global water consumption. Today, without these increased demands, 20% of the global population lack access to safe drinking water with 3.4 million people dying from water related disease. In the United States, 68% of the country is considered to be under drought conditions. With current technology and projections, it is expected that by 2030 there will be a water demand shortfall of 40%.

Large amounts of water are consumed in industrial and agricultural operations and in facility cooling operations (such as cooling towers). Many of these operations are once-through operations, where the water passes through the water system once and is then discharged, with new or fresh water being added to the system to replace the discharged water. Other operations, such as cooling towers, recycle the water a number of times prior to discharging all or a portion of the water and replacing it with fresh water. As used herein, “fresh” water refers to water that is drawn from a water supply, such as a municipal water system, nearby surface waters (ponds, lakes, or rivers), or an aquifer or well. The fresh water may be chemically treated prior to or during use, particularly for operations that recycle the water for a period of time. When discharged from these operations, the water may be sent to a wastewater treatment facility (such as through a municipal sewer system), or it may be discharged back into the surface waters, on the land, or re-injected into the aquifers. Some water discharged from these operations may be reused in other operations, such as water used in an industrial operation that is then reused in an agricultural operation. It is estimated that there are 700 billion gallons of water containing 536 billion pounds of chemicals discharged from industrial heating and cooling processes each year.

It would be beneficial to increase the amount of water from these operations that is recycled in the same operation (or recycled for extended periods of time prior to discharge) or reused in other operations to reduce the amount of fresh water needed. However, recycling and reusing the water poses specific problems because of higher inorganic and organic loads that have accumulated as a result of the primary processing and the recycling/recovery processes. Repeated recycling increases the amount of biofilm growth, fouling, and potential for corrosion, which can disrupt operations and harm equipment within the water system. Biofilms, which contain mixed communities of bacteria that adhere to surfaces, such as pipe walls, of components within the fluid system, can be particularly problematic and difficult to remove. The bacteria in underlying layers of biofilms continue to reproduce and create a dense bacterial cluster. As these biofilm layers form they also accumulate other inorganic and organic debris, increasing in size and restricting flow with the fluid system and causing blockages, which can result in increasing operating costs (such as pumping requirements) and maintenance costs for the fluid system.

Typically, the water is treated with various chemical biocides to disinfect the water to prevent or remove biofilms and other unwanted biological growth. However, these treatments increase the potential for generating harmful disinfection by-products (“DBPs”). Water treated with chemical biocides while being used in industrial operations or in heating/cooling operations may be discharged for agricultural irrigation or into various receiving waters (surface waters or sewer systems for treatment at a wastewater treatment facility), which increases the risk of exposure to DBPs to the human population through tactile contact, inhalation of volatiles, and ingestion of contaminated drinking water, produce, or livestock. DBPs include trihalomethanes (“THMs”) that form in water when chlorine or bromine (used as chemical biocides) reacts with dissolved organic carbon. THMs are a group of four toxic compounds (trichloromethane (chloroform), bromodichloromethane (BDCM), dibromochloromethane (DBCM), tribromomethane (bromoform)) suspected of causing cancer, liver and kidney damage, central nervous systems disorders, retarded fetal growth, birth defects, and possibly miscarriage. In Canada in 2011, 700 cancer cases were thought to be caused by THM exposure. Associated medical expenses are estimated approximately $140 million per year. The EPA has determined that each fatal case of cancer in the United States as a result of DBP exposure can cost as much as $5.6 million per patient and $587,500 per patient for non-fatal cases. It is clear that as the demand for treated, recycled, and reused water will increase over the coming decades, so the possibilities for human exposure to these harmful DBPs will also increase.

There are also problems associated with micro and macro pollutants in both recycled and once-through water used in industrial, agriculture, and even domestic operations. The macro pollutants are usually contaminants that exist in concentrations in the ppm range or greater and include chlorides, phosphates, and nitrates that can be effectively removed using conventional waste water treatment systems. Micro pollutants, on the other hand, represent a different problem. These are contaminants such as PCB's, phthalates, BTEX, and polybrominates diphenylesters from industrial processes; antibiotics, nonylphenol ethoxylates, and ethinyl estradiol from consumer products; DDT, tributyltin, and Triclosan from non-agricultural biocides; THM's and haloacetic acids from industrial processes and metabolites from all of the above products. Unlike the macro pollutants, these compounds exist in the pg/liter and ng/liter concentrations and are not removed by traditional wastewater treatment processes; as a result, they get re-introduced and multiplied, by repeated reuse cycles.

There are several known treatments for these types of pollutants, but these are not without their own drawbacks. For example, ozone has been found to be the most effective oxidizing treatment rendering many of these harmful micro pollutants into harmless products. However, ozone does not have any residual presence in water allowing for the proliferation of bacteria and other microorganism species in the post treated water resulting in biofouling of the surfaces of the anthropogenic water handing and sensitive heat transfer surfaces of most industrial systems. Treatment of the ozonated water with an oxidizer such as chlorine would control the biological contamination; however, this action increases the potential to form harmful THMs and other DBPs. Chlorine will oxidize many of the micro pollutants found in the post treated water and it will maintain a residual that can control biological agents in the anthropogenic water handing system. Unfortunately chlorine treatment will produce chlorinated by-products and THM's with many of the different micro pollutants. Chlorine dioxide, unlike chlorine does not form chlorinated by-products with micro pollutants and will provide residual disinfection; however, it is only effective against selective phenols and amines which limits the areas where it can be used.

It is also known that amoebas may be used to treat certain bacterial populations in water. Amoebas are single celled organisms that may be free living or parasitic. Amoebas reproduce by binary fission and multiple fission. During binary fission, the amoeba divides to produce two daughter cells. Multiple fission usually occurs under unfavorable environmental conditions and fission results in many daughter amoebas. Some amoebas are able to alternate between proliferating, feeding trophozoite and dormant cyst life stages. Amoebas move within their environments by extension of their cytoplasm. This cytoplasm is also used to trap smaller protists and bacteria where digestive enzymes are then secreted to facilitate digestion. Some bacteria such as Legionella have evolved to utilize certain amoebas, such as Hartmannella, as hosts in which they replicate. One unique amoeba that has the ability to destroy this and other bacteria which would normally invade is Willaertia magna. Dey at al. (Dey R., Bodennec J., Mameri M., and Pernin P. found that some free-living freshwater amoebae differ in their susceptibility to the pathogenic bacterium Legionella pneumophila. FEMS Microbial Lett 290 (2009) 10-17). It has been demonstrated that one Willaertia magna amoeba strain served as an ineffective host to one Legionella pneumophila bacteria strain; bacterial counts were significantly reduced in the co-culture. Additionally, U.S. Pat. No. 8,168,167 discloses the use of certain strains of amoeba genus Willaertia to treat Legionella pneumophila in drinking and industrial water. The '167 patent discloses that the Willaertia amoeba can be used as a disinfecting agent by phagocytizing and destroying Legionella pneumophila and resisting the cytotoxic effects of the bacteria. They are also able to eliminate other amoebic vectors (the free amoebas within the water that are carriers of the Legionella bacteria) which would normally allow the bacteria to escape biocide treatment. Planktonic and biofilm biomonitoring in a domestic water system showed that chlorine and chlorine dioxide were effective at removing the bacterial flora; however, amoeba in their cystic form were still able to harbor bacteria during conventional chemical treatments. After residual effects of the chemical treatments were gone, the bacteria were able to leave the amoeba and quickly re-colonize the water system. The '167 patent discloses that the use of Willaertia magna as a disinfection alternative would provide a non-chemical treatment option that would attack bacteria as well as the other amoeba hosts which protect them in harsh environments. The '167 patent discloses adding the amoeba in the form of a suspension of vegetative or cystic form or a spray in the form or a suspension of cysts and using biofilms in the water system as a site of development and growth of the amoeba. A significant drawback of the technology disclosed in the '167 patent is that the treatment amoeba cannot be grown on-site for delivery to the water system and must have a fresh solution delivered on a weekly basis, which makes this technology impractical. Additionally, it is problematic to rely on biofilms to develop the amoeba for treatment because the biofilms may grow so large and may accumulate other inorganic and organic debris that they restrict flow with the fluid system or they break off of pipe walls or other water system components and cause blockages.

It is also known to use bacteria grown on-site in bioreactors for various types of water treatments. There are several known types of bioreactors utilized to cultivate organisms include Stirred Tanks, Airlifts, Packed Beds, Fluidized Beds, Photobioreactors, Membrane Bioreactors, Rotary Drums, and Mist Bioreactors. Stirred tank bioreactors have low investment and operating costs, but foaming can be a problem in the system. Airlifts have low energy requirements and are easy to scale up, but capital costs are high and efficiency of mixing is low. Packed beds have low operating costs and are suitable for handling foam, but temperature control is poor and the unit is difficult to service and clean. Fluidized beds do not require much energy and have low shear rates, but a larger vessel size is required and internal components tend to erode. Photobioreactors maintain good temperature control and have high productivity, but the capital costs are high and shear force is a problem. Membrane bioreactors are compact and produce a high quality effluent, but there are aeration limits and the membrane can become quickly polluted. Rotary drums are effective at mixing, but the fill volume is low and operation is difficult. Mist bioreactors are low cost and do not have oxygen transfer limitation, but the system is designed only for specific cultures. Bioreactors are disclosed in U.S. Pat. Nos. 6,335,191; 7,081,361; 7,635,587; 8,093,040; and 8,551,762, for example. These and other known bioreactors are not suitable for growing more complex microorganisms, such as amoebas, which are more temperature sensitive than most bacteria used in water treatment and which require strict and consistent conditions including appropriate temperature, pH, and nutritional requirements to convert to and remain in the feeding trophozoite stage.

There is a need for an effective system and method for growing amoebas on-site for use as a biocidal water treatment to remove biofilms and other microbiological growth without the use of harsh chemicals that will both prolong the amount of time the water can be recycled in various operations prior to being discharged (to save on the amount of fresh water needed) and reduce the amount of DBPs being discharged from these operations.

### SUMMARY OF THE INVENTION

This invention provides a system and method to grow amoebas at a treatment site for use in treating industrial and commercial process water, wastewater, heating and cooling water, and other water to reduce microbiological growth and biofilms within the water and water systems. According to one preferred embodiment of the invention, an on-site biogenerator is used to grow amoeba, which are then added in a live, vegetative state into water flowing through a water system to destroy algae and harmful bacteria within the water system and to control and remove biofilm growth within the water system without requiring the use of chemical biocidal treatments. A preferred embodiment of a biogenerator according to the invention includes temperature controls that allow the device to be cooled to accommodate ambient room temperatures or down to liquid nitrogen temperatures. Another preferred embodiment of a biogenerator according to the invention includes a growth vessel or tank comprising a recirculation tube attached to a centrifugal pump that enables controlled mixing of gas (oxygen or if needed CO₂) within the growth vessel to optimize amoeba growth. Another preferred embodiment of a biogenerator according to the invention includes a feedback temperature control system and pH monitor unit to maintain optimum growth conditions within the growth vessel.

A preferred method of growing treatment amoebas according to the invention comprises periodically adding an amount of amoeba stock slug dose or pellets, water, and nutrients to a growth vessel, allowing the amoeba to grow for a period of time (residence time) within the growth vessel, and then discharging a solution containing treatment amoeba from the growth vessel. Another preferred method of growing treatment amoebas according to the invention comprises one or more of the following: adding gas (air, oxygen or CO₂) to the growth vessel during the residence time, the amount of gas added may be periodically adjusted during the residence time, monitoring the pH and periodically adjusting the pH as needed, and monitoring the temperature and periodically adjusting the temperature as needed. These steps aid in providing improved growth conditions within the growth vessel to optimize amoeba growth. Most preferably, the amoebas are grown to give a concentration of 5×10⁸ amoebas per liter of treatment solution before discharging the solution from the growth vessel.

A preferred method of treating water in a water system to remove and control the growth of algae, bacteria, and biofilms within the water system comprises providing an on-site biogenerator for growing treatment amoeba on-site, at or near a location within the water system where a solution containing treatment amoeba will be added to the water within the water system, periodically discharging an amoeba solution into the water, and allowing the water containing the treatment amoebas to contact components in the water system for a period of time. The use of an on-site generator significantly reduces costs and improves effectiveness by eliminating the need to ship, store, and continuously replenish a supply of concentrated liquid stock solutions of treatment amoeba, which are typically short lived and cannot be shipped long distances or stored for long periods of time; thereby, on site amoeba significantly reduce costs and improve effectiveness. The use of amoebas, which are naturally occurring and do not have any negative environmental impact, to treat the water in the water system eliminates or reduces the need for chemical treatment products and allows the water to be reused and recycled for longer durations before the water is discharged and replaced with fresh make-up water.

The preferred system and method according to the invention allow for growth and delivery of a precise dose of a natural biocidal organism to destroy harmful bacteria and prevent associated damage in anthropogenic water handling systems. The system is unique in that the amoeba will target planktonic as well as sessile organisms. The system utilizes the full potential of the amoeba in that it grows them to their optimal concentration depending on the system being treated on site. There is no delivery or transport time needed, resulting in many healthier, feeding organisms being delivered to the water system being treated. This process reduces or eliminates the need for dangerous chemicals to be shipped and stored, minimizing the risks associated to workers in the vicinity. The system is self-dependent and delivers the amoebas on an as-needed, timed basis. Additionally, minimal supervision is needed for operation and maintenance.

### BRIEF DESCRIPTION OF THE DRAWINGS

The system and method of the invention are further described and explained in relation to the following drawings wherein:

FIG. 1 is a perspective view of one preferred embodiment of biogenerator for growing amoebas according to the invention;

FIG. 2 is a front elevation view of the biogenerator for growing amoebas of FIG. 1;

FIG. 3 is a side elevation view of the biogenerator for growing amoebas of FIG. 1.

### DESCRIPTION OF THE PREFERRED EMBODIMENTS

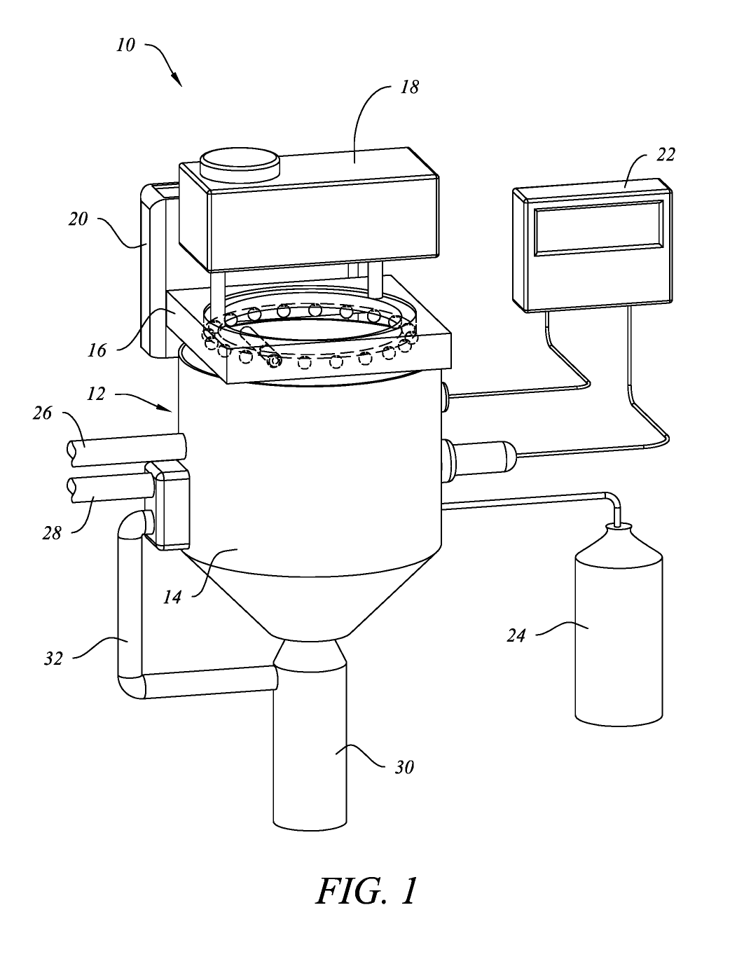

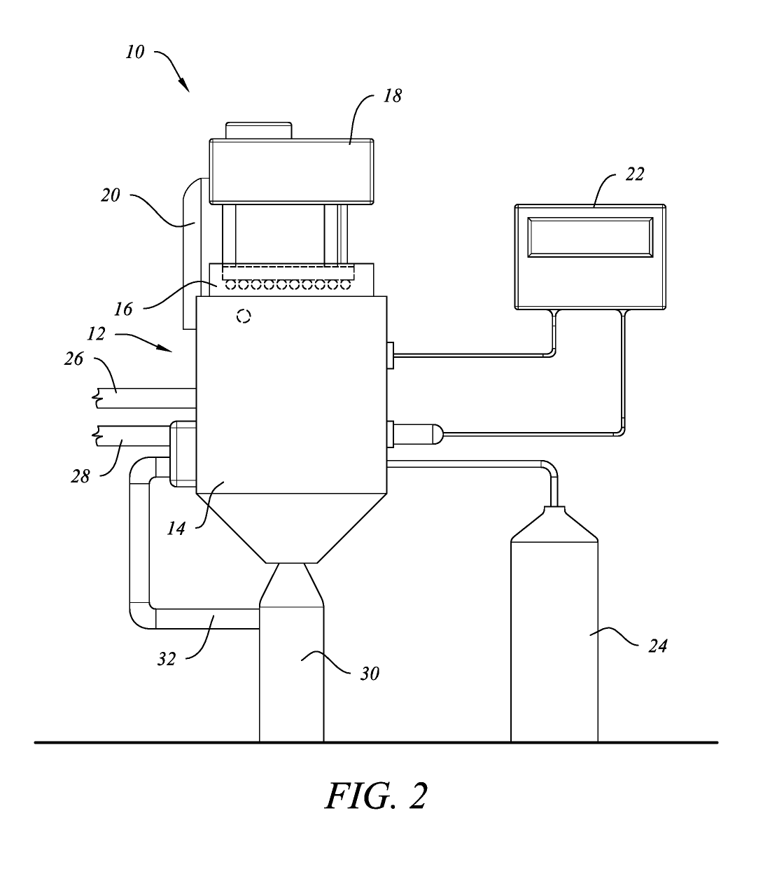

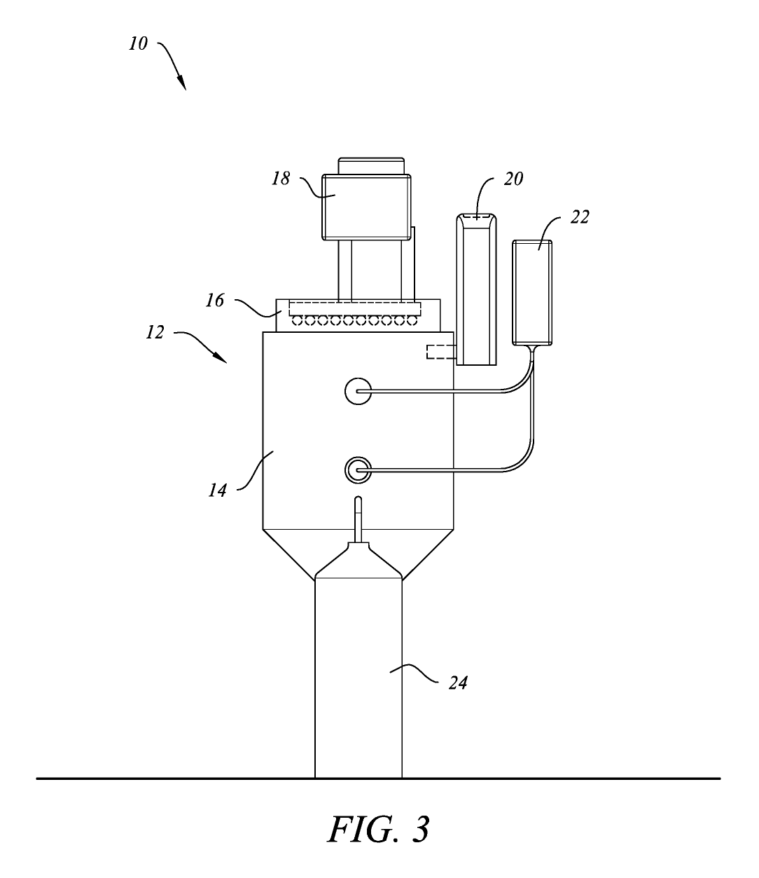

Referring to FIGS. 1-3, one preferred embodiment of a treatment system 10 for growing treatment amoebas is depicted. Treatment system 10 preferably comprises at least one biogenerator unit 12 that is in fluid communication with a water system. Amoeba solution is periodically discharged into the water of the system for treatment.

Biogenerator unit 12 preferably comprises a growth tank or growth chamber 14, an insulation chamber/amoeba starter material feeder 16, a N₂ reservoir 18, a growth media/nutrient feeder 20, a temperature and pH monitor 22, and a pressurized gas cylinder 24. Cylinder 24 may contain O₂ or CO₂. Alternatively, two cylinders 24 may be used, one containing O₂ and the other containing CO₂. An inlet 26 for adding water to the growth tank 14, a discharge outlet 28 for discharging an amoeba solution from the growth tank to the water system to be treated after an amoeba generation cycle is completed (at the end of a predetermined biogenerator or growth tank residence time), and a controller (which may be incorporated into temperature and pH controller unit 22 or may be a separate controller) are also preferably included in biogenerator unit 12. These and other components of the biogenerators disclosed in U.S. Pat. Nos. 6,335,191; 7,081,361; 7,635,587; 8,093,040; and 8,551,762, which are incorporated herein by reference, are also preferably included in biogenerator unit 12 as will be understood by those of ordinary skill in the art. The biogenerator units 12 may be battery operated, to eliminate the need for an additional external power source or outlet at the site of treatment.

The starter amoeba (which may be in the form of a liquid stock dose or pellets (such as Willaertia magna commercially available from ATCC or additional biological vendors, manufacturers, or suppliers) from insulation chamber 16 and nutrients (such as a nitrogen source, carbon source, buffer salts, mineral salts, and metals) from nutrient feeder 20 are added to the biogenerator growth tank 14. The preferred starter amoeba include Willaertia and/or other bacteria consuming amoeba, but other types of amoeba that are capable of acting as a biocide may be used if applicable. Nutrients from the nutrient feeder 20 are added to the growth tank 14 to provide a food source and promote amoeba growth during a generation cycle. Water is also added to growth tank 14 through an inlet. Most preferably, biogenerator unit 12 is connected to a source of fresh water (such as a municipal water line) or relatively uncontaminated water through the inlet with a valve controlled by a controller to add water according to predetermined cycle times or by manual input to the controller. Water may also be manually added to growth tank 14. Water may be added all at once at the beginning of a generation cycle or may be added in smaller amounts periodically during a generation cycle. The amoeba will grow inside the growth tank 14 for at least 12 to 36 hours, preferably 24 hours before being discharged through an outlet to feed into water system. Most preferably, the biogenerator unit 12 will provide a concentration of 5×10⁸ amoebas per liter +/−2 logs before discharging the solution from the growth vessel 14. The amoeba treatment solution discharged from the biogenerator 12 to the water system with each dose preferably comprises amoeba in the trophozoite form, which are capable of acting as a biocide for planktonic and sessile bacterial and biofilm immediately upon discharge into the water system and without producing harmful waste by-products.

Biogenerator unit 12 also preferably comprises a recirculation tube 32 attached to a centrifugal pump 30, which enables controlled mixing of introduced O₂ or CO₂ within the growth vessel 14. A controller operates the centrifugal pump to open or close a valve delivering gas from a pressurized cylinder 24 as needed to as needed to optimize amoeba growth within the vessel 14. A temperature and pH monitor and control unit 22 also receive input from the growth tank regarding the temperature and pH of the amoeba solution within the growth tank 14 during a generation cycle. The temperature and pH readings may be displayed so that a user can manually adjust settings to alter the temperature or pH, but most preferably, the monitor 22 or controller are programmed to automatically adjust operational settings for biogenerator unit 12 to alter the temperature or pH as needed to optimize amoeba growth. Liquid nitrogen from N₂ reservoir 18 is fed to the insulation chamber 16 containing the amoeba starter material to keep the starter material cold. Temperature control unit 22 will monitor and adjust the temperature as needed. Various pH modifying agents, such as acids or bases, may be added to growth tank 14 in response to pH readings of the solution within the growth vessel 14 by the pH monitor 22. For Willaertia amoeba, the conditions within the growth vessel 14 are preferably maintained to provide a temperature between about 33° C. and 37° C., a pH between about 6 and 7, and an oxygen level between about 5 and 25 ppm, in order to optimize growth of the treatment amoeba. Other treatment amoebas may have different optimization conditions as will be understood by those of ordinary skill in the art.

Most preferably, the water system being treated is supplied with a fresh dose of treatment amoeba from the biogenerator unit 12 at least once every 24 hours, but dosing cycles between every 12 to 36 hours may also be used. Dosing cycles preferably occur during normal operations of the water system and are suspended if the water system is shut-down, for maintenance or repair or if the flow of water through the water system device is substantially reduced.

Biogenerator unit 12 is preferably configured to hold enough amoeba starter material to supply growth vessel 14 with a suitable amount of starter material for around a 30 day treatment cycle before the starter material needs to be replenished. Depending on the duration of each generation cycle (residence time within growth vessel prior to discharging the treatment solution), the biogenerator unit 12 can provide around 15 to 60 doses of treatment solution during a 30 day treatment cycle. Growth vessel 14 is preferably sized to hold between 1 and 3 liters of amoeba solution just prior to discharging to the water system being treated. Typically, a single biogenerator unit will be sufficient to treat most water systems as long as the concentration of amoeba remains 10³.+/−1 log. If larger quantities of water require treatment, treatment system 10 may be easily scaled up by adding additional biogenerator units in parallel and/or by increasing the size of the growth vessel (with corresponding increases in the amount of starter amoeba, nutrients, and water added) to provide larger quantities of amoeba treatment solution. Additionally, more than one biogenerator unit may be used if it is desired to feed different amoebas at different rates. For example, one biogenerator may be configured to discharge a solution comprising Willaertia magna once every 12 hours and a second biogenerator may be configured to discharge a solution comprising a second amoeba species once every 24 hours. Treatment system 10 may be scaled up to provide around 250 gallons of amoeba treatment solution per day.

The use of an on-site biogenerator allows the amoeba solution to be added to the water system to be treated in a vegetative state and allows large quantities of amoebas to be added with each dosing cycle, thereby increasing the effectiveness of the treatment and reducing costs and losses associated with shipping and storage of off-site generated amoebas. Most preferably, a fresh batch of high concentration of amoeba (5×10⁸ amoebas per liter) will be grown and administered to the water system being treated on a daily basis, although other dosing cycles may be used. The amoeba will have a short lifetime in the conditions of an anthropogenic water system, so repeated and frequent dosing with an amoeba treatment solution are important to ensure biological efficacy in in consumption of both bacteria and biofilm within the water system.

Most preferably, a viable amoeba starting culture (or starter material) is maintained for a treatment cycle of around 30 days, which is the typical service cycle for traditional chemical treatment programs, before the amoeba starter material, nutrients, and other consumables within the treatment system 10 need to be replenished. Treatment cycles may be periodically repeated or may be run in a substantially continuous manner (with shut-downs for maintenance and replenishment as needed). Factors that will influence the viability of the amoeba starting culture include form factor (cryo-preserved, freeze dried, pelletized, or liquid feed), storage temperature, and starting life stage (cyst or trophozoites). Optimization of these factors may vary with different amoeba species, but for Willaertia is it preferred to use a cryo-preserved form for the starting culture, 33° C. to 37° C. for storage temperature, and trophozoite as the starting life stage.

Many of the water systems to be treated with the system and method of the invention will involve water that is cycled through the water system multiple times or that is recycled from one water system to another water system. This makes the water quality highly variable, with possible high concentrations of calcium, magnesium, iron, copper, chlorides, and phosphates. Temperature may vary as well, all of which may impact the amoeba life cycle and impact how the amoeba will grow and transition through life stages in these different environments. Most preferably, doubling times will be measured in different water chemistries to determine the best amoeba species and operational parameters for particular water systems, to optimize amoeba growth, and determine the duration of treatment of each dose of amoeba treatment solution once it is discharged into the water system. Operational parameters of the water system (temperature, pH, etc.) may be modified to increase growth rate and treatment duration (duration of viability of the amoeba once discharged into the water system) for each treatment dose.

A method according to a preferred embodiment of the invention comprises growing amoebas in a biogenerator (most preferably a biogenerator according to the preferred system described above) for at least 12 to 36 hours and preferably around 24 hours, discharging or feeding the vegetative amoebas from the biogenerator (preferably according to the dosing cycles and amounts described with system 10 above) to a water system to be treated, monitoring and maintaining an optimized temperature for an amoeba starter material to be fed into the biogenerator, monitoring operating parameters of the biogenerator to maintain conditions that allow the amoeba to grow within a growth tank during a generation cycle, altering operating parameters as needed to maintain the parameters within desired ranges. The operating parameters include one or more of the following: dissolved oxygen level, CO₂ level, temperature, and pH. The amoebas used are as described above. Data from the monitoring steps may be used to adjust operating parameters, such as through the addition of pH adjusting additives or added aeration to increase oxygen levels, to bring the parameters within desired ranges. Data from the monitoring may be automatically sent to a control system or may be manually input into a control system to alter components or to change the operating parameters if they are not within desired ranges. The changes or alterations to the operating parameters may be carried out automatically by an automated control system or may be manually made.

Most preferably, the biogenerator is capable of producing a plurality of doses of amoeba treatment solution over a treatment cycle, with each dose being discharged to the water system to be treated once every 12 to 36 hours, and preferably around once every 24 hours. The treatment solution circulates through the water system or is otherwise allowed to contact components within the water system to allow the amoeba to consume planktonic and sessile bacteria and biofilms within the water system. A preferred method also comprises monitoring operating parameters of the water system, such as temperature and pH, and adjusting those parameters to optimize viability of the amoeba once discharged into the water system. A preferred method further comprises sampling the water from the water system, measuring doubling times of the amoeba in the system water and selecting or adjusting one or more of the following parameters based on the doubling time measurement: the amount of amoeba treatment solution added the water system, an operating parameter of the water system (such as pH or temperature), the species of amoeba in the treatment solution, or dosing cycle time.

Those of ordinary skill in the art will also appreciate upon reading this specification and the description of preferred embodiments herein that modifications and alterations to the device may be made within the scope of the invention and it is intended that the scope of the invention disclosed herein be limited only by the broadest interpretation of the appended claims to which the inventors are legally entitled.

We claim:

1. A treatment system for treating water in a water system to reduce or remove biological contaminants, the treatment system comprising a biogenerator for generating a plurality of doses of an amoeba treatment solution from an amoeba starter material, the biogenerator comprising a growth tank and an outlet for discharging each dose of amoeba treatment solution to the water system at predetermined dosing cycle intervals.

2. The treatment system according to claim 1 wherein the amoeba treatment solution comprises one or more of bacteria-consuming amoeba.

3. The treatment system according to claim 1 wherein the dosing cycle interval is between once every 12 hours and once every 36 hours during normal operations.

4. The treatment system according to claim 1 wherein each dose of amoeba treatment solution comprises at least 5×10⁸ amoebas per liter of solution.

5. The treatment system according to claim 4 wherein substantially most of the amoebas in each dose of treatment solution are in the trophozoite form prior to discharging the treatment solution to the water system.

6. The treatment system according to claim 1 wherein the amoebas in the treatment solution are capable of consuming planktonic and sessile bacteria and biofilms within the water system.

7. The treatment system according to claim 1 wherein the biogenerator further comprises an insulation chamber and a reservoir for liquid nitrogen.

8. The treatment system according to claim 7 further comprising a monitoring system for monitoring one or more operating parameters of the biogenerator to determine if they are within a predetermined range or above or below a predetermined threshold for that parameter.

9. The treatment system according to claim 8 wherein the operating parameters comprise temperature or pH within the growth tank.

10. The treatment system according to claim 9 wherein the operating parameters are adjustable if they are outside the predetermined range or above or below the predetermined threshold to optimize growing amoeba growing conditions within the growth tank.

11. The treatment system according to claim 9 wherein the predetermined range of temperatures is between about 33° C. and 37° C.

12. The treatment system according to claim 9 wherein the predetermined range of pH values is between about 6 and 7.

13. The treatment system according to claim 7 wherein the biogenerator further comprises a feeder for holding an amount of amoeba starter material and the feeder is maintained at a temperature between about 5 and 25 ppm.

14. The treatment system according to claim 7 further comprising one or more sources of pressurized gas in fluid communication with the growth tank.

15. The treatment system according to claim 14 wherein the pressurized gas is CO₂ or O₂ or both.

16. The treatment system of claim 1 wherein the treatment system is configured to discharge a dose of amoeba treatment solution for each dosing cycle interval that is up to 250 gallons of amoeba treatment solution every 24 hours.

17. The treatment system of claim 16 wherein the treatment system comprises a plurality of biogenerators, each discharging a dose of amoeba treatment solution for each dosing cycle interval, with the total volume of all such doses being equivalent up to 250 gallons of amoeba treatment solution every 24 hours.

18. A method for treating water in a water system to reduce or remove biological contaminants, the method comprising the steps of:
providing a biogenerator comprising a growth tank in fluid communication with the water system;
providing an amoeba starter material;
controlling the temperature of the amoeba starter material to maintain a temperature around a predetermined temperature or range of temperatures;
generating a dose of treatment solution comprising vegetative amoeba in the growth tank from the amoeba starter material; and
discharging the dose of treatment solution from the growth tank to the water system after a predetermined biogenerator residence time.

19. The method of claim 18 further comprising repeating the generating and discharging steps of claim 18 to periodically generate and discharge a new dose of treatment solution at predetermined time intervals.

20. The method of claim 18 further comprising controlling the temperature of the treatment solution in the growth tank during the biogenerator residence time to maintain the temperature around a predetermined temperature or range of temperatures.

21. The method of claim 20 further comprising controlling the pH of the treatment solution in the growth tank during the biogenerator residence time to maintain the pH around a predetermined pH value or range of values.

22. The method of claim 21 further comprising controlling the amount of dissolved gas within the treatment solution in the growth tank during the biogenerator residence time to maintain the level of dissolved gas around a predetermined level or range of levels.

23. The method of claim 18 wherein the amoeba starter material comprises one or more of bacteria-consuming amoebas.

24. The method of claim 20 wherein the predetermined range of temperatures is between about 33° C. and 37° C.

25. The method of claim 21 wherein the predetermined range of pH values is between about 6 and 7.

26. The method of claim 22 wherein the dissolved gas is CO₂ and the predetermined range of levels is between about 5 and 25.

27. The method of claim 23 further comprising the steps of:
obtaining a sample of water from the water system;
measuring doubling times of the one or more amoeba species from the treatment solution in the sample; and
selecting one or more other amoeba species based on the measured doubling times and adding or replacing the amoeba starter material with such one or more other amoeba species.

28. The method of claim 24 further comprising the steps of:
obtaining a sample of water from the water system;
measuring properties of the water sample; and
providing an amoeba starter material comprising one or more amoeba species based on the measured properties.

29. The method of claim 28 wherein the measured properties are one or more of concentrations of calcium, magnesium, iron, copper, chlorides, and phosphates.

30. The method of claim 18 further comprising adding pressurized CO₂ or O₂ or both to the growth tank during the generating step.
